(19) United States
(12) Patent Application Publication
(10) Pub. No.: US 2018/0161281 A1
(43) Pub. Date: Jun. 14, 2018

(54) EXTENDED RELEASE PHARMACEUTICAL COMPOSITION OF MIXED AMPHETAMINES

(71) Applicant: Lupin Atlantis Holdings SA, Somerset, NJ (US)

(72) Inventors: Velayutham Kanniyappan, Somerset, NJ (US); James Garegnani, Somerset, NJ (US)

(21) Appl. No.: 15/832,432

(22) Filed: Dec. 5, 2017

Related U.S. Application Data
(60) Provisional application No. 62/431,796, filed on Dec. 8, 2016.

(51) Int. Cl. A61K 9/50; A61K 9/48; A61K 31/137

(52) U.S. Cl. CPC A61K 9/5073; A61K 9/5047; A61K 31/137; A61K 9/501; A61K 9/4808; A61K 9/5026

(57) ABSTRACT

A long-acting pharmaceutical composition comprising at least one amphetamine salt is provided. The composition comprises an immediate release component, a delayed release component, and a sustained release component comprising one or more amphetamine salts. The sustained release component comprises a sustained release layer and a delayed release layer, such that the delayed release layer is coated onto the sustained release layer. The time (Tₘₐₓ) to achieve Cₘₐₓ after administration of said composition is between 7-10 hours.

### BACKGROUND OF THE INVENTION

### (a) Field of the Invention

The present invention is directed to a long-acting pharmaceutical composition comprising at least one amphetamine salt. The invention is further directed to use of said composition for treating conditions responsive to administration of amphetamine salts including Attention deficit hyperactivity disorder (ADHD).

### (b) Description of the Related Art

Attention deficit hyperactivity disorder (ADHD) is a developmental disorder characterized by symptoms such as impulsiveness, hyperactivity and/or inattentiveness. Hyperactivity is common among children with ADHD but tends to disappear during adulthood. However, over half of children with ADHD continue to have some symptoms of inattention throughout their lives.

Stimulant medications are widely used as a pharmacological treatment for ADHD. Stimulants, in the short term, have been found to be safe in the appropriately selected patient and appear well tolerated over five years of treatment. Several pharmaceutical agents approved in the US for use in treatment of ADHD are primarily affectors of the dopamine or norepinephrin neural pathways. Approved agents include salts and isomers of amphetamine and methylphenidate, dextroamphetamine prodrug, lisdexamfetamine dimesylate, and atomoxotine.

One of the challenges of treating ADHD and other CNS stimulant responsive conditions is delivering and maintaining an effective concentration in patients throughout the day and in particular, in the morning hours when cognitive abilities and concentration are needed for school or work and in the late afternoon or evening when students often do homework.

Adderall™ is an immediate release composition, which includes a mixture of four amphetamine salts: dextroamphetamine sulfate, dextroamphetamine saccharate, amphetamine aspartate monohydrate and amphetamine sulfate. This combination of amphetamines is indicated for the treatment of ADHD in children from 3-10 years of age.

The immediate release formulations, however, require twice daily administration, causing problems with compliance, are time-consuming, inconvenient and may be problematic for those children having difficulties in swallowing tablet formulations.

Various long-acting formulations were developed and are now available that have been shown in clinical trials to be effective for from 8-14 hours (Brams et al., Current Medical Research and Opinion, vol. 26 no. 8, pgs 1809-1825, August 2010).

Adderall XR™, Strattera™, Concerta™, Metadae CD™, Ritalin™, Vyvanse™ and Focalin XR™ are some of the medications currently approved by the U.S. Food and Drug Administration (FDA) for the treatment of ADHD. Strattera™ is an atomoxetine (a norepinephrine reuptake inhibitor) medication. Long acting stimulant preparations, such as Adderall XR™ and Concerta™ (methylphenidate), are designed to provide a duration of effect up to 12 hours. Adderall XR™ met the need for a dosage form, which can be administered once, in place of the two oral doses which are needed using the conventional drug delivery formulations of the prior art. See U.S. Pat. Nos. 6,322,819; 6,605,300 and Reissue Pat. Nos. RE41,148; RE42,096. Adderall XR™ is a mixed amphetamine salts medication.

Clinicians, however, have noted that a proportion of patients treated with formulations like Adderall XR™ and Strattera™ require additional treatment with a short-acting stimulant to extend the daily therapeutic effect. For patients taking long-acting stimulant formulations who require duration of clinical benefit beyond 10-12 hours, clinicians have augmented the morning long-acting formulation, typically at 8-10 hours post-dose, with a dose of the same immediate-release (IR) medication. Typically, the dose of the IR medication is smaller than the long-acting dose. This augmentation strategy is most relevant to the “longer day demands” of adult and adolescents, rather than school age, pediatric patients.

U.S. Pat. Nos. 9,028,868; 9,119,809 and 9,289,394 discloses a once daily delayed release dosage comprising a core comprising a therapeutic amount of a central nervous system stimulant and at least one pharmaceutically acceptable excipient; a sustained release layer coating the core; and a delayed release layer coating the sustained release layer. The patents teach that the dosage form exhibits a lag period of at least 5 hours during which the plasma concentration of amphetamine or its salt is less than 10% of the maximum concentration (Cₘₐₓ) and it achieves the Cₘₐₓ between 12 and 19 hours (Tₘₐₓ) after administration. Such dosage form is thus suitable only for evening time administration due to its typical plasma profile and as a result, it exhibits therapeutic effects from the next day's morning onwards.

U.S. Pat. Nos. 8,846,100; 9,173,857 and Pub. No. 2016/0015642 A1 disclose a multiple pulsed dose drug delivery system for amphetamine salts, comprising amphetamine salt covered with an immediate-release coating, amphetamine salt covered with a delayed release coating, and amphetamine salt covered with a sustained release coating and a delayed coating, wherein the sustained release coating is applied over the delayed release coating. The patents and publication further teach that a sustained release formulation constructed with a delayed release coating overlaying a sustained release coating results in a Tₘₐₓ that is too early after administration to a patient to result in a composition that meets the longer-day requirements for the treatment of ADHD.

In summary, the known long-acting formulations of amphetamine salts suggest that formulations containing a delayed release coating over the sustained release coating either results in a Tₘₐₓ that is too early to render it suitable for longer-day ADHD treatment or results in a Tₘₐₓ that is too late to provide round-the-clock therapeutic benefit to ADHD patients starting immediately after administration.

Therefor a need exists for a once-daily, long-acting oral composition that provides effective treatment of ADHD, without supplementation, for patients with longer day demands (e.g., 14-16 awake hours) immediately, without any significant lag between the administration time and time of relief from the ADHD symptoms, and without the augmentation of an immediate-release dose.

### SUMMARY OF THE INVENTION

The present invention provides the following aspects, subject-matters and preferred embodiments, which respectively taken alone or in combination, further contribute to solving the object of the present invention.

An improved long-acting pharmaceutical composition of amphetamine salts has been developed. The composition is made by three types of components comprising one or more amphetamine salts. The composition provides extended release of amphetamine in the gastrointestinal tract for a duration of at least 12 hours and fills the need for once-daily and a day-long treatment of ADHD.

In one aspect, the present invention provides a pharmaceutical composition comprising: a combination of (a) an immediate release component, (b) a delayed release component, and (c) a sustained release component, wherein the sustained release component comprises a delayed release coating and a sustained release coating arranged such that the sustained release coating is layered onto the delayed release coating.

In another aspect, the present invention provides a pharmaceutical composition comprising:
(a) a first population of bead comprising—
   (i) at least one amphetamine salt layered onto or incorporated into a core,
   (ii) a delayed release coating layered onto the core, and
   (iii) a coating comprising at least one amphetamine salt layered onto the delayed release coating;
and
(b) a second population of bead comprising—
   (i) at least one amphetamine salt layered onto or incorporated into a core,
   (ii) a sustained release coating layered onto the core, and
   (iii) a delayed release coating layered onto the sustained release layer.

In another aspect, the time (Tₘₐₓ) to achieve Cₘₐₓ upon administration of the composition is between 7 hours to about 10 hours.

One or more amphetamine salt is/are selected from the group consisting of amphetamine sulfate, amphetamine aspartate or its monohydrate, dextroamphetamine sulfate, dextroamphetamine saccharate, and mixtures thereof.

In a further aspect, the first population of bead provides first pulsed release of the amphetamine or its salt and the second population of bead provides second pulsed release or sustained release of the amphetamine or its salt.

In a further aspect, the sustained release coating is pH-independent and the delayed release coating is pH dependent.

In a further aspect, both the delayed release bead and sustained release bead comprise an enteric coating.

In a further aspect, the delayed release bead and the sustained release bead comprise different enteric coatings.

In a further aspect, the delayed release layer can include a pH dependent polymer or copolymer that is insoluble in aqueous medium at pH lower than 5.5. Such a delayed release layer can include, but is not limited to cellulose acetate phthalate, cellulose acetate trimaletate, hydroxypropyl methylcellulose phthalate, polyvinyl acetate phthalate, acrylic polymers, polyvinyl acetaldiethylamino acetate, hydroxypropyl methylcellulose acetate succinate, cellulose acetate trimellitate, shellac, methacrylic acid copolymers, Eudragit® L30D, Eudragit® L100, Eudragit® FS30D, Eudragit® S100 or combinations of any thereof. The delayed release layer can also include a plasticizer. In an embodiment, the delayed release layer includes methacrylic acid copolymer, talc and a plasticizer, preferably, triethyl citrate.

In a further aspect, the sustained release layer includes a water-insoluble and water-permeable polymer and can further include a water soluble polymer. In certain embodiments, the sustained release layer includes, but is not limited to polyvinyl acetate, cellulose acetate, cellulose acetate butyrate, cellulose acetate propionate, ethyl cellulose, fatty acids, polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylam ides, polymethacrylam ides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin, esters thereof, alkyl alcohols, waxes, zein (prolamine from corn), and aqueous polymeric dispersions such as EUDRAGIT® RS and RL30D, EUDRAGIT® NE30D, AQUACOAT®, SURELEASE®, KOLLICOAT® SR30D, cellulose acetate latex or a combination of any thereof. In an embodiment, the sustained release layer comprises ethyl cellulose and talc.

In a further aspect, the composition comprises about 12.5 mg, 18.75 mg, 25 mg, 31.25 mg, 37.5 mg, 43.75 mg, 50 mg, 62.5 mg or 75 mg of amphetamine salt.

In a further aspect, the ratio of the amount of amphetamine salt layered onto or incorporated into a core of the first population of bead to that layered onto a delayed release coating of the first population of bead to that layered onto or incorporated into a core of the second population of bead is about 2:1:2

In a further aspect, administration of a 37.5 mg dose of the pharmaceutical composition to a human patient results in a dextroamphetamine Cₘₐₓ of about 30-70 ng/ml after about 6-9 hours (Tₘₐₓ) and a levoamphetamine Cₘₐₓ of about 5-25 ng/ml after about 6-9 hours (Tₘₐₓ).

In a further aspect, the area under the curve from time 0 to the last measured time (AUC₀₋ₗₐₛₜ) after administration of a 37.5 mg dose of the pharmaceutical composition to a human patient is about 850-1250 ng·hr/ml for dextroamphetamine and about 250-550 ng·hr/ml for levoamphetamine.

In a further aspect, the area under the curve from time 0 to time infinity (AUC_{0-inf}) after administration of a 37.5 mg dose of the pharmaceutical composition to a human patient is about 850-1250 ng·hr/ml for dextroamphetamine and about 250-550 ng·hr/ml for levoamphetamine.

In a further aspect, the pharmaceutical composition further comprises a protective layer between the core and coating, between any two coatings or between core and all coatings.

The pharmaceutical compositions disclosed herein can be in the form of coated beads, or they can be compressed into tablet or minitablet form. The beads or minitablets can then be apportioned in single dose amounts into water soluble gelatin capsules, or into a liquid or gel suspension for administration.

In another aspect, the present invention provides a method of treating or preventing ADHD comprising administering to a subject in need thereof the pharmaceutical composition as substantially described throughout the specification.

Still other aspects and advantages of the invention will be apparent from the following detailed description of the invention.

### DETAILED DESCRIPTION OF THE INVENTION

The present invention provides a pharmaceutical composition of one or more amphetamine salts suitable for once-daily administration. The composition provides round-the clock treatment of ADHD without need for augmentation to an additional immediate-release dose.

The invention provides a long-acting amphetamine pharmaceutical composition, which includes an immediate release component, a delayed release component and a sustained release component, to meet the therapeutic needs of ADHD patients with longer-day demands. The present invention fills the need for once-daily longer-day treatment of ADHD by providing an amphetamine pharmaceutical composition that is bioequivalent to an equal dosage of Adderall XR™ followed by an immediate release amphetamine composition 8 hours later.

It was found that addition of a delayed release formulation having a lag time of about 8 hours to Adderall XR™ which simulates to the administration of an immediate release amphetamine composition 8 hours after the administration of Adderall XR™, cannot meet the recognized need for a once-daily long-acting amphetamine composition that meets a patient's longer day requirements. In contrary to the teachings of the prior art, the inventors have surprisingly found that the composition of the present invention can mimic the bioavailability of an equivalent total amphetamine dosage provided by Adderall XR™ followed by an immediate release amphetamine composition 8 hours later.

The composition of the present invention comprises a combination of (a) an immediate release component, (b) a delayed release component, and (c) a sustained release component which comprises a delayed release coating and a sustained release coating arranged such that the sustained release coating is layered onto the delayed release coating. It was found that with such unique composition, it is possible to achieve a Tₘₐₓ that is suitable to meet the longer-day requirements for the treatment of ADHD.

In an embodiment, the sustained release component of the present invention comprises at least one amphetamine salt layered onto, or incorporated into, a core; a sustained release coating layered onto the amphetamine core; a delayed release coating layered onto the sustained release coating; and, optionally, a protective coating.

The three components comprising the extended release amphetamine composition of the invention release doses of the active ingredients at varying, pre-determined times to provide for full day treatment (i.e., about 14 hours to about 16 hours) of conditions such as ADHD. A treatment for ADHD, which can be delivered in a single dosage is especially beneficial to adolescents and adults who typically have longer daily waking hours compared to children.

The compositions of the present invention comprise an immediate release component, a delayed release component, and a sustained release component.

In an embodiment, both delayed release and sustained release components comprise an enteric coating.

In an embodiment, the immediate release component, the delayed release component and the sustained release component each contain equal amounts of active ingredient.

In a preferred embodiment, the immediate release component, the delayed release component and the sustained release component contains active ingredient in amount ratio of about 2:1:2 respectively.

In one embodiment, the immediate release, the delayed release and the sustained release components of the composition are present on the same core. In another embodiment, the immediate release and delayed release components are present on different cores. In a further embodiment, the delayed release and sustained release components are present on different cores. In a preferred embodiment, the immediate release and delayed release component are present on one core and the sustained release component is present on a second core.

In yet another embodiment, the amphetamine salt is coated onto a core. In a further embodiment, the amphetamine salt is incorporated into a core.

It is contemplated that compositions of the present invention can include a combination of the hereinabove referred to cores (one or more cores that include three components on the same core, one or more cores that include two of the three components on the core, and one or more cores that include one of the three components on the core).

A pharmaceutical composition according to the present invention includes:
(a) an immediate release bead comprising an amphetamine salt;
(b) a delayed release bead comprising an amphetamine salt; and
(c) a sustained release bead comprising an amphetamine salt;

wherein the delayed release bead provides first release rate of the mixed amphetamine salt and the sustained release bead provides second release rate of the mixed amphetamine salt.

A pharmaceutical composition of the present invention provides a patient with at least about 14 hours to about 16 hours of effective therapy for attention deficit hyperactivity disorder (ADHD).

In an embodiment, the dextroamphetamine Cₘₐₓ after administration of a 37.5 mg amphetamine pharmaceutical composition to a human patient is about 30-70 ng/ml after about 6-9 hours (Tₘₐₓ).

In a further embodiment, the dextroamphetamine area under the curve from time 0 to the last measured time (AUC₀₋ₗₐₛₜ) after administration of a 37.5 mg amphetamine pharmaceutical composition to a human patient is about 850-1250 ng·hr/ml.

In a further embodiment, the dextroamphetamine area under the curve from time 0 to time infinity (AUC_{0-inf}) after administration of a 37.5 mg dose of the pharmaceutical composition to a human patient is about 850-1250 ng·hr/ml.

In an embodiment, the levoamphetamine Cₘₐₓ after administration of a 37.5 mg amphetamine pharmaceutical composition to a human patient is about 5-25 ng/ml after about 6-9 hours (Tₘₐₓ).

In a further embodiment, the levoamphetamine area under the curve from time 0 to the last measured time (AUC₀₋ₗₐₛₜ) after administration of a 37.5 mg amphetamine pharmaceutical composition to a human patient is about 250-550 ng·hr/ml.

In a further embodiment, the levoamphetamine area under the curve from time 0 to time infinity (AUC_{0-inf}) after administration of a 37.5 mg dose of the pharmaceutical composition to a human patient is about 250-550 ng·hr/ml.

In a further embodiment, a protective layer is provided over at least one enteric coating. In another embodiment, a protective layer is provided between the amphetamine salt core or coating, at least one enteric coating and sustained release coating. A protective layer can also be provided over the sustained release coating according to the present invention.

In an embodiment, the amphetamine salt is selected from the group consisting of dextroamphetamine sulfate, dextroamphetamine saccharate, amphetamine aspartate or its monohydrate, amphetamine sulfate, and mixtures thereof. In a more particular embodiment, the amphetamine salt is a mixture of dextroamphetamine sulfate, dextroamphetamine saccharate, amphetamine aspartate monohydrate, and amphetamine sulfate.

The present invention encompasses methods for treating ADHD, which comprise administering the amphetamine salt pharmaceutical composition of the present invention to a patient suffering from ADHD.

U.S. Pat. No. 8,846,100 suggests that the amount of enteric coating (10-15% w/w) for the delayed release component results in undesired premature leakage of the drug from the delivery system into the upper gastrointestinal tract causing reduced drug delivery at the desired, more distal location in the gastrointestinal tract. In contrary to such teaching, it was surprisingly found that use of an enteric coating in amount 10-20% w/w in the pharmaceutical composition of the invention in fact achieved the desired release profile, and as a result may provide full beneficial therapeutic activity at the desired time.

The term “bead” refers to a discrete component of a dosage form. For example, a capsule shell is filled with a plurality of beads. As used herein, bead encompass any discrete component of a dosage form including minitablet, pellet and granule.

“Immediate” and “delayed” release refer to the onset of release in relationship to administration of the drug. “Immediate” means that the release of drug begins very soon, within a relatively short time after administration, e.g. a few minutes or less. “Delayed” means that the release of drug is postponed, and begins or is triggered some period of time after administration (e.g., the lag time), typically a relatively long period of time, e.g. more than one hour.

“Sustained” refers to the period of ongoing release, and means that the delivery of drug goes on (it continues or is sustained) for an extended period of time after initial onset, typically more than one hour, whatever the shape of the dose release profile. For example, the drug release is sustained between a maximum and minimum value (more than zero) for some relatively long period of time. This release may be at a constant dose, or at a dose which diminishes over time.

The term “about” means the referenced numeric indication plus or minus 10% of that referenced numeric indication.

Drug release and drug release profiles are measures or representations of the manner and timing by which a formulation releases or delivers active ingredients (drug) to a receiving environment (e.g. the stomach, intestines, etc.) upon administration. Various methods are known for evaluating drug release and producing release profiles, including in vitro tests which model the in vivo behaviour of a formulation. These include USP dissolution testing.

A plasma profile is a measure or representation of the dose or level of active ingredient (drug) in the bloodstream of a mammal, e.g. a patient receiving a drug formulation. Upon release of a drug from a formulation, e.g. into the gut of a mammal, the amount of drug that is present in the bloodstream over time can be determined.

A drug release profile may be designed to produce a desired or targeted plasma profile. Often, but not necessarily, a plasma profile will mimic a release profile. For example, it might be expected that a sustained release of drug would more likely produce a sustained dose in the plasma, or that a pulsed release would produce a pulsed (peak and valley) plasma profile. Other factors may also play a role, such as bio-absorption, bioavailability, and first pass effect. The plasma profile produced by a particular release profile may also vary from patient to patient.

Measures of bioavailability well known in the art include the area under the plasma concentration-time curve (AUC), the concentration maximum (Cₘₐₓ), and the time to Cₘₐₓ, known as Tₘₐₓ.

AUC is a measurement of the area under the plasma concentration-time curve, and is representative of the amount of drug absorbed following administration of a single dose of a drug (Remington: The Science and Practice of Pharmacy, (Alfonso R. Gennaro ed. 2000), page 999).

Cₘₐₓ, is the maximum plasma concentration achieved after oral drug administration (Remington, page 999). An oral drug administration results in one Cₘₐₓ, but may result in greater than one “peak plasma concentration” or “plasma concentration peak” (for example, following the administration of a pulsed dose formulation).

Tₘₐₓ, is the amount of time necessary to achieve the Cₘₐₓ, after oral drug administration, and is related to the rate of absorption of a drug (Remington, page 999).

Bioequivalence is the absence of a significantly different rate and extent of absorption in the availability of the active ingredient when administered at the same dose under similar conditions. Bioequivalence can be measured by pharmacokinetic parameters such as, for example, AUC and Cₘₐₓ.

A drug delivery system of the invention typically may comprise a core seed or matrix, which may or may not be loaded with drug, and one or more coating layers comprising drug, and/or comprising a layer have release characteristics which control the onset and release characteristics of the drug. An exemplary core is a sugar core. Exemplary matrixes include hydrophilic matrixes. Polymers useful for forming a hydrophilic matrix include hydroxypropyl methylcellulose (HPMC), hydroxypropyl cellulose (HPC), poly(ethylene oxide), poly(vinyl alcohol), xanthan gum, carbomer, carrageenan, and zooglan. Other similar hydrophilic polymers may also be employed.

The present invention comprises a core or starting seed, which can be either a prepared or commercially available product. The cores or starting seeds can be sugar spheres, spheres made from microcrystalline cellulose and any suitable drug crystals.

The materials that can be employed in making drug-containing beads are any of those commonly used in pharmaceutics and should be selected on the basis of compatibility with the active drug and the physicochemical properties of the beads. The additives except active drugs are chosen from the known and conventional pharmaceutical excipients including binders, disintegrants, fillers, surfactants, solubilizers, stabilizers etc.

The core particles may have a diameter in the range of about 50-1500 microns; preferably 100-800 microns.

The core particles can then be coated in a fluidized bed apparatus with an alternating sequence of coating layers.

A core can include, for example, an active agent, a disintegrant, a pore forming agent, and a binder. An exemplary core includes about 10-25% w/w active agent. The drug containing core can be made by a variety of processes known in the art, including wet granulation, extrusion, and spheronization.

The core may be coated directly with a layer or layers of at least one amphetamine salts and/or the amphetamine salt may be incorporated into the core material. Amphetamine salts contemplated to be within the scope of the present invention include amphetamine base and salts thereof. Preferred pharmaceutically active amphetamine salts include dextroamphetamine sulfate, dextroamphetamine saccharate, amphetamine aspartate monohydrate and amphetamine sulfate.

A protective layer may be added on top of the pharmaceutical active containing layer and also may be provided between active layers. A separation or protective layer may be added onto the surface of the active-loaded core, and then the enteric delayed pulsed or sustained release layer is coated thereupon. Another active layer is preferably added to the enteric delayed release layer to deliver an initial dose.

The protective coating layer may be applied immediately outside the core, either a drug-containing core or a drug layered core, by conventional coating techniques such as pan coating or fluid bed coating using solutions of polymers in water or suitable organic solvents or by using aqueous polymer dispersions. Suitable materials for the protective layer include cellulose derivatives such as hydroxyethyl cellulose, hydroxypropyl cellulose, hydroxypropyl methylcellulose, polyvinylpyrrolidone, polyvinylpyrrolidone/vinyl acetate copolymer, ethyl cellulose aqueous dispersions (AQUA COAT®, SURELEASE®), EUDRAGIT® RL 30D, OPADRY® and the like. The suggested coating levels are from 1-8% w/w, preferably 2-5% w/w.

In an embodiment, two layers are coated cover the core. The first layer is a sustained release layer and the outer layer is a delayed release layer that is optionally pH dependent. In a particular embodiment, the sustained release layer comprises about 3-10% w/w ethyl cellulose and about 0.5-2% w/w talc, and the delayed release layer comprises about 10-20% w/w methacrylic acid copolymer, about 0.5-2% w/w talc and about 0.5-2% w/w triethyl citrate.

The enteric delayed release or sustained release coating layer is applied onto the cores with or without seal coating by conventional coating techniques, such as pan coating or fluid bed coating using solutions of polymers in water or suitable organic solvents or by using aqueous polymer dispersions. Suitable coaters are well known in the art. For example, any commercially available pH-sensitive polymer can be used. With such a polymer, the pharmaceutical active is not released in the acidic stomach environment of approximately below pH 4.5, but is not limited to this value. The pharmaceutical active should become available when the pH sensitive layer dissolves at the greater pH; after a certain delayed time; or after the unit passes through the stomach.

Enteric coatings suitable for use in the composition of the invention can be pH-dependent or pH-independent. Preferably, the enteric coatings for both the delayed release component and sustained release component are pH dependent. A pH dependent coating is activated to release drug within a known pH range, which typically is matched to the local pH of the environment where delayed release is desired. Exemplary pH dependent coatings include cellulose acetate phthalate, cellulose acetate trimellitate, hydroxypropyl methylcellulose phthalate, polyvinyl acetate phthalate, carboxymethylethyl cellulose, co-polymerized methacrylic acid/methacrylic acid methyl esters such as, for instance, materials known under the trade name EUDRAGIT®) L12.5, L100, or EUDRAGIT® S12.5, S100 or similar compounds used to obtain enteric coatings. Aqueous colloidal polymer dispersions or re-dis persions can be also applied, e.g. EUDRAGIT® L 30D-55, EUDRAGIT®) L100-55, EUDRAGIT®) S100, EUDRAGIT® preparation 4110D (Rohm Pharma), AQUA TERIC®, AQUACOAT® CPD 30 (FMC); KOLLICOAT MAE® 30D and 30DP (BASF); EASTACRYL® 30D (Eastman Chemical).

Exemplary sustained release coatings can include one or more water soluble and/or water insoluble polymers selected from polyvinyl acetate, cellulose acetate, cellulose acetate butyrate, cellulose acetate propionate, ethyl cellulose, fatty acids, polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose, hydroxymethyl cellulose and hydroxypropyl methylcellulose, sodium carboxymethyl cellulose, methylcellulose, hydroxyethyl methylcellulose, hydroxypropyl methylcellulose, polyacrylates such as carbomer, polyacrylamides, polymethacrylam ides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin, esters thereof, alkyl alcohols, waxes, zein (prolamine from corn), and aqueous polymeric dispersions such as EUDRAGIT® RS and RL30D, EUDRAGIT® NE30D, AQUACOAT®, SURELEASE®, KOLLICOAT® SR30D, and cellulose acetate latex.

Plasticizers suitable for use in the present invention include, but are not limited to, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, low molecular weight poly(ethylene glycol), citrate ester-type plasticizers, triacetin, propylene glycol and glycerin. Such plasticizers can also include ethylene glycol, 1,2-butylene glycol, 2,3-butylene glycol, styrene glycol, diethylene glycol, triethylene glycol, tetraethylene glycol and other poly(ethylene glycol) compounds, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, acetyl triethyl citrate, tributyl citrate and allyl glycolate.

The compositions of the present disclosure can also include one or more functional excipients such as lubricants, thermal lubricants, antioxidants, buffering agents, alkalinizing agents, binders, diluents, sweeteners, chelating agents, colorants, flavorants, surfactants, solubilizers, wetting agents, stabilizers, hydrophilic polymers, hydrophobic polymers, waxes, lipophilic materials, absorption enhancers, preservatives, absorbents, cross-linking agents, bioadhesive polymers, retardants, pore formers, and fragrance.

The composition, preferably in beadlet form, can be incorporated into hard gelatin capsules, either with additional excipients, or alone. Typical excipients to be added to a capsule formulation include, but are not limited to: fillers such as microcrystalline cellulose, soy polysaccharides, calcium phosphate dihydrate, calcium sulfate, lactose, sucrose, sorbitol, or any other inert filler. In addition, there can be flow aids such as fumed silicon dioxide, silica gel, magnesium stearate, calcium stearate or any other material imparting flow to powders. A lubricant can further be added if necessary by using polyethylene glycol, leucine, glyceryl behenate, magnesium stearate or calcium stearate.

In an embodiment, the pharmaceutical composition comprises:
(a) a first population of bead comprising—
   (i) about 10-20% w/w of at least one amphetamine salt layered onto or incorporated into a core,
   (ii) a delayed release coating layered onto the core comprising about 10-20% w/w methacrylic acid copolymer, about 0.5-2% w/w talc and about 0.5-2% w/w plasticizer, and
   (iii) a coating comprising about 5-10% w/w at least one amphetamine salt layered onto the delayed release coating;
and
(b) a second population of bead comprising—
   (i) about 15-25% w/w at least one amphetamine salt layered onto or incorporated into a core,
   (ii) a sustained release coating layered onto the core comprising about 3-10% w/w ethyl cellulose and about 0.5-2% w/w talc, and
   (iii) a delayed release coating layered onto the sustained release layer comprising about 10-20% w/w methacrylic acid copolymer, about 0.5-2% w/w talc and about 0.5-2.5% w/w plasticizer.

In another embodiment, the pharmaceutical composition comprises:
(a) a first population of bead comprising—
(i) about 10-20% w/w of at least one amphetamine salt layered onto or incorporated into a core,
(ii) delayed release coating layered onto the core comprising about 15-25% w/w methacrylic acid copolymer, about 1.0-2.5% w/w talc and about 1-2.5% w/w plasticizer, and
(iii) a coating comprising about 5-10% w/w at least one amphetamine salt layered onto the delayed release coating;
and
(b) a second population of bead comprising—
(i) about 15-25% w/w at least one amphetamine salt layered onto or incorporated into a core,
(ii) a sustained release coating layered onto the core comprising about 5-15% w/w ethyl cellulose and about 0.1-2% w/w talc, and
(iii) a delayed release coating layered onto the sustained release layer comprising about 15-25% w/w methacrylic acid copolymer, about 0.5-15% w/w talc and about 0.5-2.5% w/w plasticizer

wherein the time to achieve Cₘₐₓ is in between 7-10 hours after administration of the composition.

The composition can be incorporated into a tablet, in particular by incorporation into a tablet matrix, which rapidly disperses the particles after ingestion. In an embodiment, a tablet according to the present invention can be constructed in one, two or three layers, wherein the immediate release component is dry blended, and the delayed release and the sustained release components are wet granulated. The tablet is then formed in a one layer, two layer or a three layer compression. In some embodiments, the composition is a water soluble capsule that contain coated beads.

It will be appreciated that the multiple dosage form of the present invention can deliver rapid and complete dosages of amphetamine salts to achieve the desired levels of the drug in a recipient over the course of about 14 hours to about 16 hours with a single oral administration.

### Example 1: Extended Release Capsule of Mixed Amphetamine Salts

TABLE 1
Immediate release beads

| Sr. | | Quantity | |
|---|---|---|---|
| No. | Material | mg/Unit | % w/w |
| 1 | Sugar spheres 30/35 | 102.50 | 59.16 |
| 2 | Dextroamphetamine Saccharate | 6.25 | 3.61 |
| 3 | Amphetamine Aspartate Monohydrate | 6.25 | 3.61 |
| 4 | Dextroamphetamine Sulfate | 6.25 | 3.61 |
| 5 | Amphetamine Sulfate | 6.25 | 3.61 |
| 6 | Hydroxypropyl methylcellulose 2910 | 37.50 | 21.65 |
| 7 | Hydroxypropyl methyl cellulose 2910 | 8.25 | 4.76 |
| | (Seal coat) | | |
| 8 | Purified water | Q.S. | Q.S. |

| | | |
|---|---|---|
| TOTAL | 173.25 | 100.00 |

Procedure:

A drug solution was prepared by dissolving Hydroxypropyl methylcellulose 2910, Dextroamphetamine Saccharate, Amphetamine Aspartate Monohydrate, Dextroamphetamine Sulfate and Amphetamine Sulfate in water. Sugar spheres were loaded in a fluid bed processor equipped with a Wurster column. The prepared drug solution was sprayed on to the sugar spheres under suitable process condition. The drug loaded pellets were dried and seal coated with HPMC.

TABLE 2
Delayed release beads

| Sr. | | Quantity | |
|---|---|---|---|
| No. | Material | mg/Unit | % w/w |
| 1 | Immediate release beads of Table 1 | 86.63 | 73.25 |
| 2 | Eudragit L 30-D 55 | 21.70 | 18.35 |
| 3 | Talc | 2.15 | 1.82 |
| 4 | Triethyl citrate | 2.15 | 1.82 |
| 5 | Hydroxypropyl methylcellulose 2910 | 5.63 | 4.76 |
| 6 | Purified water | Q.S. | Q.S. |

| | | |
|---|---|---|
| TOTAL | 118.26 | 100.00 |

Procedure:

The immediate release beads (as per Table 1) were coated with an Eudragit L 30 D-55 dispersion. The beads prepared as per Table 1 were loaded in a fluid bed processer. The coating dispersion was prepared by dispersing triethyl citrate, talc and Eudragit L 30 D-55. The prepared dispersion was sprayed on to the immediate release beads under suitable process condition. The delayed release beads were dried and seal coated with Hydroxypropyl methylcellulose 2910.

TABLE 3
Immediate release/Delayed release beads

| Sr. | | Quantity | |
|---|---|---|---|
| No. | Material | mg/Unit | % w/w |
| 1 | Delayed release beads of Table 2 | 118.26 | 79.09 |
| 2 | Dextroamphetamine Saccharate | 3.13 | 2.09 |
| 3 | Amphetamine Aspartate Monohydrate | 3.13 | 2.09 |
| 4 | Dextroamphetamine Sulfate | 3.13 | 2.09 |
| 5 | Amphetamine Sulfate | 3.13 | 2.09 |
| 6 | Hydroxypropyl methylcellulose 2910 | 18.75 | 12.54 |
| 7 | Purified water | Q.S. | Q.S. |

| | | |
|---|---|---|
| TOTAL | 149.53 | 100.00 |

Procedure:

The delayed release beads as per Table 2 were coated with a drug solution prepared by dissolving Hydroxypropyl methylcellulose 2910 and all four Amphetamine salts in water. The delayed release beads from Table 2 were loaded in a fluid bed processer. The prepared drug solution was sprayed on to the delayed release beads under suitable process conditions to have the immediate release drug coating (IR/DR beads).

TABLE 4
Sustained release beads

| | |
|---|---|
| Sr. | Quantity |

| No. | Material | mg/Unit | % w/w |
|---|---|---|---|
| 1 | Immediate release beads of Table 1 | 86.63 | 69.96 |
| 2 | Surelease E - 7 - 19040 | 6.80 | 5.49 |
| 3 | Talc | 1.80 | 1.45 |
| 4 | Eudragit FS 30-D | 24.00 | 19.38 |
| 5 | Talc | 2.30 | 1.86 |
| 6 | Triethyl citrate | 2.30 | 1.86 |
| 7 | Purified water | Q.S. | Q.S. |

| | | |
|---|---|---|
| TOTAL | 123.83 | 100.00 |

Procedure:

The immediate release beads as per Table 1 were coated with Surelease dispersion to have a sustained release layer. The beads from Table 1 were loaded in a fluid bed processer. The coating dispersion was prepared by dispersing talc and Surelease in water. The prepared dispersion was sprayed on to the immediate release beads under suitable process condition. The sustained release beads were dried and further enteric coating was done using Eudragit FS 30-D coating dispersion. An Eudragit FS 30-D coating dispersion was prepared by dispersing triethyl citrate, talc and Eudragit FS 30-D.

Finally, the enteric coated pellets were dried and encapsulated along with immediate release/delayed release beads prepared as per Table 3.

### Example 2: Extended Release Capsule of Mixed Amphetamine Salts

TABLE 5
Immediate release beads

| Sr. | | Quantity | |
|---|---|---|---|
| No. | Material | mg/Unit | % w/w |
| 1 | Sugar spheres 30/35 | 175.00 | 80.53 |
| 2 | Dextroamphetamine Saccharate | 6.25 | 2.88 |
| 3 | Amphetamine Aspartate Monohydrate | 6.25 | 2.88 |
| 4 | Dextroamphetamine Sulfate | 6.25 | 2.88 |
| 5 | Amphetamine Sulfate | 6.25 | 2.88 |
| 6 | Hydroxypropyl methylcellulose 2910 | 10.90 | 5.02 |
| 7 | Opadry Beige YS-1-17274 | 6.4 | 2.95 |
| 8 | Purified water | Q.S. | Q.S. |

| | | |
|---|---|---|
| TOTAL | 217.30 | 100.00 |

Procedure:

A drug solution/suspension was prepared using Hydroxypropyl methylcellulose 2910, Dextroamphetamine Saccharate, Amphetamine Aspartate Monohydrate, Dextroamphetamine Sulfate and Amphetamine Sulfate in water. Sugar spheres were loaded in a fluid bed processor equipped with a Wurster column. The prepared drug solution was sprayed on to the sugar spheres under suitable process condition. The drug loaded pellets were dried and seal coated with Opdary Beige YS-1-17274.

TABLE 6
Delayed release beads

| Sr. | | Quantity | |
|---|---|---|---|
| No. | Material | mg/Unit | % w/w |
| 1 | Immediate release beads of Table 5 | 108.65 | 72.99 |
| 2 | Eudragit L 30-D 55 | 29.88 | 20.07 |
| 3 | Talc | 2.99 | 2.01 |
| 4 | Triethyl citrate | 2.99 | 2.01 |
| 5 | Hydroxypropyl methylcellulose 2910 | 4.35 | 2.92 |
| 6 | Purified water | Q.S. | Q.S. |

| | | |
|---|---|---|
| TOTAL | 148.85 | 100.00 |

Procedure:

The immediate release beads (as per Table 5) were coated with an Eudragit L 30 D-55 dispersion. The beads prepared as per Table 5 were loaded in a fluid bed processer. The coating dispersion was prepared by dispersing triethyl citrate, talc and Eudragit L 30 D-55. The prepared dispersion was sprayed on to the immediate release beads under suitable process condition. The delayed release beads were dried and seal coated with Hydroxypropyl methylcellulose 2910.

TABLE 7
Immediate release/Delayed release beads

| Sr. | | Quantity | |
|---|---|---|---|
| No. | Material | mg/Unit | % w/w |
| 1 | Delayed release beads of Table 6 | 148.85 | 84.17 |
| 2 | Dextroamphetamine Saccharate | 3.125 | 1.77 |
| 3 | Amphetamine Aspartate Monohydrate | 3.125 | 1.77 |
| 4 | Dextroamphetamine Sulfate | 3.125 | 1.77 |
| 5 | Amphetamine Sulfate | 3.125 | 1.77 |
| 6 | Hydroxypropyl methylcellulose 2910 | 11.00 | 6.22 |
| 7 | Opadry Beige YS-1-17274 | 4.50 | 2.54 |
| 8 | Purified water | Q.S. | Q.S. |

| | | |
|---|---|---|
| TOTAL | 176.85 | 100.00 |

Procedure:

The delayed release beads as per Table 6 were coated with a drug suspension/solution prepared using Hydroxypropyl methylcellulose 2910 and all four Amphetamine salts in water. The delayed release beads from Table 6 were loaded in a fluid bed processer. The prepared drug suspension/solution was sprayed on to the delayed release beads under suitable process conditions. The drug loaded pellets were dried and seal coated with Opdary Beige YS-1-17274 to have the immediate release drug coating (IR/DR beads).

TABLE 8
Sustained release beads

| | |
|---|---|
| Sr. | Quantity |

| No. | Material | mg/Unit | % w/w |
|---|---|---|---|
| 1 | Immediate release beads of Table 5 | 108.65 | 55.28 |
| 2 | Surelease E-7-19040 | 21.72 | 11.05 |
| 3 | Eudragit FS 30-D (based on solids) | 45.63 | 23.22 |
| 4 | Talc | 18.25 | 9.29 |
| 5 | Triethyl citrate | 2.28 | 1.16 |
| 6 | Purified water | Q.S. | Q.S. |

| | | |
|---|---|---|
| TOTAL | 196.53 | 100.00 |

Procedure:

The immediate release beads as per Table 5 were coated with Surelease dispersion to have a sustained release layer. The beads from Table 5 were loaded in a fluid bed processer. The coating dispersion was prepared by dispersing Surelease in water. The prepared dispersion was sprayed on to the immediate release beads under suitable process condition. The sustained release beads were dried and further enteric coating was done using Eudragit FS 30-D coating dispersion. Eudragit FS 30-D coating dispersion was prepared by dispersing triethyl citrate, talc and Eudragit FS 30-D.

Finally, the enteric coated pellets were dried and encapsulated along with immediate release/delayed release beads prepared as per Table 7.

What is claimed is:

1. A pharmaceutical composition comprising:
(a) a first population of bead comprising—
(i) at least one amphetamine salt layered onto or incorporated into a core,
(ii) a delayed release coating layered onto the core, and
(iii) a coating comprising at least one amphetamine salt layered onto the delayed release coating;
and
(b) a second population of bead comprising—
(i) at least one amphetamine salt layered onto or incorporated into a core,
(ii) a sustained release coating layered onto the core, and
(iii) a delayed release coating layered onto the sustained release layer;
wherein the time to achieve Cₘₐₓ is in between 7-10 hours after administration of the composition.

2. The pharmaceutical composition of claim 1, wherein the at least one amphetamine salt is selected from the group consisting of amphetamine sulfate, amphetamine aspartate or its monohydrate, dextroamphetamine sulfate, dextroamphetamine saccharate, and mixtures thereof.

3. The pharmaceutical composition of claim 1, wherein the ratio of the amount of amphetamine salt layered onto or incorporated into a core of the first population of bead to that layered onto a delayed release coating of the first population of bead to that layered onto or incorporated into a core of the second population of bead is about 2:1:2.

4. The pharmaceutical composition of claim 1, wherein the first population of bead provide pulsed release of the amphetamine or its salt and the second population of bead provides sustained release of the amphetamine or its salt.

5. The pharmaceutical composition of claim 1, wherein the sustained release coating is pH-independent and the delayed release coating is pH dependent.

6. The pharmaceutical composition of claim 1, wherein the first delayed release bead and the second delayed release bead comprise an enteric coating.

7. The pharmaceutical composition of claim 1, wherein the first delayed release bead and the second delayed release bead comprise different enteric coatings.

8. The pharmaceutical composition of claim 1, which further comprises a protective layer between the core and coating, between any two coatings or between core and all coatings.

9. The pharmaceutical composition of claim 1, wherein the amount of at least one amphetamine salt is about 12.5 mg, 18.75 mg, 25 mg, 31.25 mg, 37.5 mg, 43.75 mg, 50 mg, 62.5 mg or 75 mg.

10. The pharmaceutical composition of claim 1, wherein administration of a 37.5 mg dose of the pharmaceutical composition to a human patient results in a dextroamphetamine Cₘₐₓ of about 30-70 ng/ml after about 6-9 hours (Tₘₐₓ).

11. The pharmaceutical composition of claim 1, wherein administration of a 37.5 mg dose of the pharmaceutical composition to a human patient results in a levoamphetamine Cₘₐₓ of about 5-25 ng/ml after about 6-9 hours (Tₘₐₓ).

12. The pharmaceutical composition of claim 1, wherein the dextroamphetamine area under the curve from time 0 to the last measured time (AUC₀₋ₗₐₛₜ) after administration of a 37.5 mg dose of the pharmaceutical composition to a human patient is about 850-1250 ng·hr/ml.

13. The pharmaceutical composition of claim 1, wherein the dextroamphetamine area under the curve from time 0 to time infinity (AUC_{0-inf}) after administration of a 37.5 mg dose of the pharmaceutical composition to a human patient is about 850-1250 ng·hr/ml.

14. The pharmaceutical composition of claim 1, wherein the levoamphetamine area under the curve from time 0 to the last measured time (AUC₀₋ₗₐₛₜ) after administration of a 37.5 mg dose of the pharmaceutical composition to a human patient is about 250-550 ng·hr/ml.

15. The pharmaceutical composition of claim 1, wherein the levoamphetamine area under the curve from time 0 to time infinity (AUC_{0-inf}) after administration of a 37.5 mg dose of the pharmaceutical composition to a human patient is about 250-550 nghr/ml.

16. A method for treating attention deficit hyperactivity disorder comprise administering the pharmaceutical composition of claim 1 to a patient suffering from said condition.

17. A pharmaceutical composition comprising:
(a) a first population of bead comprising—
(i) about 10-20% w/w of at least one amphetamine salt layered onto or incorporated into a core,
(ii) a delayed release coating layered onto the core comprising about 20% w/w methacrylic acid copolymer, about 0.5-2% w/w talc and about 0.5-2% w/w plasticizer, and
(iii) a coating comprising about 5-10% w/w at least one amphetamine salt layered onto the delayed release coating;
and
(b) a second population of bead comprising—
(i) about 15-25% w/w at least one amphetamine salt layered onto or incorporated into a core,
(ii) a sustained release coating layered onto the core comprising about 3-10% w/w ethyl cellulose and about 0.5-2% w/w talc, and
(iii) a delayed release coating layered onto the sustained release layer comprising about 10-20% w/w methacrylic acid copolymer, about 0.5-2% w/w talc and about 0.5-2.5% w/w plasticizer.
wherein the time to achieve Cₘₐₓ is in between 7-10 hours after administration of the composition.

18. The pharmaceutical composition of claim 17, wherein the ratio of the amount of amphetamine salt layered onto or incorporated into a core of the first population of bead to that layered onto a delayed release coating of the of first population of bead to that layered onto or incorporated into a core of the second population of bead is about 2:1:2.
